(19) United States
(12) Patent Application Publication
(10) Pub. No.: US 2004/0127475 A1
(43) Pub. Date: Jul. 1, 2004

(54) Apparatus and method for delivering compounds to a living organism

(75) Inventors: Gishel New, Victoria (AU); Jeffrey W. Moses, New York, NY (US); Nicholas Kipshidze, New York, NY (US); Gary S. Roubin, Jackson, WY (US); Martin B. Leon, New York, NY (US)

Correspondence Address: MICHAEL BEST & FRIEDRICH, LLP, 100 E WISCONSIN AVENUE, MILWAUKEE, WI, 53202, US

(73) Assignee: Estrogen Vascular Technology, LLC, New York, NY

(21) Appl. No.: 10/661,820

(22) Filed: Sep. 12, 2003

Related U.S. Application Data
(63) Continuation-in-part of application No. 10/260,954, filed on Sep. 30, 2002.
Application No. 10/260,954 is a division of application No. 09/943,648, filed on Aug. 30, 2001, now Pat. No. 6,471,979.
Application No. 09/943,648 is a continuation-in-part of application No. PCT/US00/35641, filed on Dec. 29, 2000.
(60) Provisional application No. 60/173,451, filed on Dec. 29, 1999.
(60) Provisional application No. 60/410,387, filed on Sep. 12, 2002.

(51) Int. Cl. A61K 31/56; A61M 31/00

(52) U.S. Cl. 514/171; 604/500

(57) ABSTRACT

A method of treating or preventing high-risk plaque is provided. The method may include applying to a medical device an effective amount of a composition comprising a sex hormone, anti-hormone, sex-hormone agonist, steroid-hormone inhibitor/antagonist (partial or full), selective estrogen receptor modulator (SERM), or a combination thereof. The medical device may be inserted into an area of a living organism that is or has a propensity to be affected by high-risk plaque.

### CROSS-REFERENCE TO RELATED APPLICATIONS

This patent application is a continuation-in-part of and claims priority to application Ser. No. 10/260,954 filed on Sep. 30, 2002, which is a divisional of application Ser. No. 09/943,648 filed on Aug. 30, 2001, which issued as U.S. Pat. No. 6,471,979 on Oct. 29, 2002, which is a continuation-in-part (CIP) of international application no. PCT/US00/35641 filed on Dec. 29, 2000, which claims the benefit of U.S. provisional application No. 60/173,451 filed on Dec. 29, 1999. This application also claims priority under 35 U.S.C. § 119(e) to co-pending U.S. provisional patent application No. 60/410,387 filed Sep. 12, 2002.

### BACKGROUND OF THE INVENTION

Vascular diseases include diseases that affect areas of a living organism relating to or containing blood vessels. For example, stenosis is a narrowing or constricting of arterial lumen or blood vessels in a living organism (e.g., a human) usually due to atherosclerosis (AS) or coronary heart disease (CHD). Restenosis is a recurrence of stenosis after a percuteneous intervention such as angioplasty and/or stenting. The underlying mechanisms of restenosis comprise a combination of effects from vessel recoil, negative vascular remodeling, thrombus formation and neointimal hyperplasia. It has been shown that restenosis after balloon angioplasty is mainly due to vessel remodeling and neointimal hyperplasia vessel recoil and after stenting is mainly due to neo-intimal hyperplasia.

Treatment for stenosis and restenosis varies. Stenosis caused by AS or CHD often forces individuals to restrict and limit their activity levels in order to avoid complications, angina, intermittent claudication, rest pain, stroke, heart attack, sudden death and loss of limb or function of a limb stemming from the stenosis. The reconstruction of blood vessels, arteries and veins may also be needed to treat individuals suffering from stenosis and restenosis. Coronary bypass can also be utilized to revascularize the heart and restore normal blood flow. In other cases, balloon angioplasty may be conducted to increase the orifice size of culprit areas. Overall, these treatments address the problems associated with stenosis, but they also create a high rate of restenosis that can result in recurrence of cardiac symptoms and mortality. Moreover, these treatments are not preventative in nature, and therefore generally are not utilized until the patient or individual has already developed stenosis.

One cause of stenosis and restenosis is atherosclerosis. Atherosclerosis affects medium and large arteries and is characterized by a patchy, intramural thickening that encroaches on the arterial lumen and, in most severe form, causes obstruction. The atherosclerotic plaque comprises an accumulation of intracellular and extracellular lipids, smooth muscle cells and connective tissue. The earliest lesion of atherosclerosis is the fatty streak that evolves into a fibrous plaque coating the artery. Atherosclerotic vessels have reduced systolic expansion and abnormal wave propagation. Treatment of atherosclerosis is usually directed at its complications, for example, angina, myocardial infarction, claudication, arrhythmia, heart failure, kidney failure, stroke, and peripheral arterial occlusion.

New and improved methods and devices are being sought for treatment and prevention of vascular diseases such as stenosis, restenosis and atherosclerosis.

### SUMMARY OF THE INVENTION

In one aspect, the invention provides a method of treating or preventing high-risk plaque. The method may include applying to a medical device an effective amount of a composition comprising a sex hormone, anti-hormone, sex-hormone agonist, steroid-hormone inhibitor/antagonist (partial or full), selective estrogen receptor modulator (SERM), or a combination thereof. The medical device may be inserted into an area of a living organism that is or has a propensity to be affected by high-risk plaque.

In another aspect, the invention provides a local-delivery device for treating or preventing high-risk plaque in a living organism. The local-delivery device includes a medical device at least partially coated with an effective dose of a composition comprising a sex hormone, anti-hormone, sex-hormone agonist, steroid-hormone inhibitor/antagonist (partial or full), selective estrogen receptor modulator (SERM), or a combination thereof. The local-delivery device may be suitable for treating or preventing high-risk plaque.

In yet another aspect, the invention provides a method of treating high-risk plaque in a living organism. The method includes applying an effective dose of a composition comprising estrogen, estradiol or a derivative thereof to a stent by chemical or physical bonding. The stent is placed at or near high-risk plaque and estrogen, estradiol or derivative thereof is released

### BRIEF DESCRIPTION OF THE DRAWINGS

FIG. 1 is a perspective view of a stent embodying the invention.

FIG. 2 is a cross-sectional view taken along line 2--2 in FIG. 1.

FIG. 3 is a perspective view of a balloon-injection catheter embodying the invention.

FIG. 4 is cross-sectional view taken along line 4--4 of FIG. 3.

FIG. 5 is a cross-sectional view taken along line 5--5 in FIG. 5, wherein the catheter is inserted into an affected area of a living organism.

FIG. 6 is a cross-sectional view taken along line 6--6 of FIG. 1.

FIG. 7 is a table illustrating photomicrographs of histological section 30 days after delivery of a) control stent b) low dose 17B-estradiol stent, and c) high dose 17B-estradiol stent illustrating intimal proliferation.

FIG. 8 is a table illustrating dosage data for studies performed in Example 2.

FIG. 9 is a table showing averages and standard deviations for the dosage per stent and dosage per unit area.

FIG. 10 shows total dosage per stent for the various stent designs, which was estimated by multiplying the average dosage per unit area by the stent surface areas.

FIG. 11 is a representation of stable plaque.

FIG. 12 is a representation of vulnerable plaque.

FIG. 13 is a representation of a vulnerable plaque, and the consequences of its rupture. Factors limiting thrombosis include high flow, fibrinolytic activity, and minor plaque disruption. A non-occlusive plaque/thrombus may be silent and result in angina, silent infarction, sudden death or acute coronary syndrome. An occlusive thrombus may also result in sudden death. Certain factors precipitate, contribute or accelerate thrombosis: inflammatory or immune response, increased platelet reactivity, decreased fibrinolytic activity and major plaque disruption.

FIG. 14 is a chart depicting angiographic and IVUS follow-up results related to Example 3.

FIG. 15 is a chart showing percentage of drug retained on stent versus time.

Other features and advantages of the invention will become apparent to those skilled in the art upon review of the following detailed description and claims. Before embodiments of the invention are explained in detail, it is to be understood that the invention is not limited in its application to the details of the composition and concentration of components set forth in the following description. The invention is capable of other embodiments and of being practiced or being carried out in various ways. Also, it is understood that the phraseology and terminology used herein is for the purpose of description and should not be regarded as limiting.

### DESCRIPTION OF THE INVENTION

The present invention provides apparatuses and methods for delivering a composition to a localized area of a living organism. The invention relates to local-delivery devices and methods for treating and preventing proliferative and atherosclerotic vascular diseases in a living organism. More particularly, the invention provides apparatuses and methods for locally delivering a sex hormone (e.g. estrogen), an anti-hormone, a sex-hormone agonist, a steroid-hormone inhibitor/antagonist (partial or full) or a selective estrogen receptor modulator (SERM), or a combination thereof, to a portion of a living organism inflicted by or susceptible to a vascular disease such as stenosis or restenosis. The local-delivery device, e.g. a stent, catheter, injection catheter, balloon or balloon-injection catheter in situ to coat the implanted stent, is inserted into an affected area of a living organism to treat or prevent the proliferative and atherosclerotic vascular disease. Balloons have been developed so that drugs can seep out though the wall without being injected.

Gender differences in cardiovascular disease have largely been attributed to the protective effects of estrogen in women; premenopausal women have a lower incidence of Coronary Heart Disease. In particular, estrogen has well-known beneficial effects on lipid profile. More importantly, estrogen may directly affect vascular reactivity, which is an important component of atherosclerosis. More particularly, many epidemiological studies suggest that estrogen replacement therapy (ERT) may be cardioprotective in postmenopausal women. The beneficial effects of these hormone therapies may also be applicable to males. Unfortunately the systemic use of estrogen has limitations due to the possible hyperplastic effects of estrogen on the uterus and breast in women, and the feminizing effects in males.

The mechanisms for these beneficial effects are probably multifactorial. Estrogen is known to favorably alter the atherogenic lipid profile and may also have a direct action on blood vessel walls. Estrogen can have both rapid and long-term effects on the vasculature including the local production of coagulation and fibrinolytic factors, antioxidants and the production of other vasoactive molecules, such as nitric oxide and prostaglandins, all of which are known to influence the development of vascular disease.

Experimental work suggests that estrogen can also act on the endothelium and smooth muscle cells either directly or via estrogen receptors in both men and women. This appears to have an inhibitory effect on many steps in the atherosclerotic process. With respect to the interventional cardiology, estrogen appears to inhibit the response to balloon injury to the vascular wall. Estrogen can repair and accelerate endothelial cell growth in-vitro and in-vivo. Early restoration of endothelial cell integrity may contribute to the attenuation of the response to injury by increasing the availability of nitric oxide. This in turn can directly inhibit the proliferation of smooth muscle cells. In experimental studies, estrogen has been shown to inhibit the proliferation and migration of smooth muscle cells in response to balloon injury. Estrogen has also proved to inhibit adventitial fibroblast migration, the mechanism involved in negative remodeling.

Effective Compositions

Sex hormones and sex-hormone agonists may be helpful in preventing and treating certain vascular diseases. Examples of suitable sex hormones include, but are in no way limited to, estrogens, progesterones, testosterones, dehydroepiandrostrones (DHEAs) and dehydroepiandrosteronesulfates (DHEAs) and derivatives thereof. Of these compounds, estrogen has proven to be the most effective in preventing and treating vascular diseases. Naturally occurring/plant estrogens or phytoestrogens including isoflavones such as genistein, daidzein and resveratrol are also useful in the treatment of vascular disease. Suitable sex-hormone agonists include, but are in no way limited to, estradiol, estrone, ethinyl estradiol, conjugated equine estrogens and derivatives thereof.

In addition, anti-hormones and steroid-hormone inhibitors/antagonist (partial or full) may be effective in preventing vascular diseases. Anti-hormones inhibit or prevent the usual effects of certain other hormones, thereby increasing the relative effectiveness of hormones that are not being inhibited or prevented by these anti-hormones. Anti-hormones effective in preventing vascular diseases include, but are not limited to, anti-estrogens (e.g. Faslodex), anti-androgens (e.g. cyproterone acetate) and anti-testosterone (e.g. anti-testosterone wild-type Fab fragment and mutant Fab fragments). Examples of steroid-hormone inhibitors/antagonist (partial or full) include, but are not limited to, aminogluthemide, anastrazole and letrozole.

Selective estrogen receptor modulators (SERMS), including but not limited to raloxifene, tamoxifen, tibolone and idoxifene, may also be effective in treating or preventing vascular diseases such as stenosis and restenosis.

These compounds are generally found in a powdered form. In order to apply the compound to a local-delivery device or to locally inject the compound into an affected area, the powder is generally mixed with a solution of saline or ethanol. This facilitates coating the local-delivery devices or injecting the composition as described below. The composition can also be mixed into another solution, gel or substance to control the rate of release from the stent and into the tissue.

Local-Delivery Systems

Local delivery of the above-listed compositions in the exact area of disease or potential disease avoids the negative systemic effects these compounds can produce when administered generally. The devices can be inserted into arteries, both coronary and otherwise. Oral use of conjugated equine estrogen in combination with a progestin may have effects on the coagulation pathways that attenuate the benefits that may potentially occur to a vascular wall. In addition, hyperplastic effects of estrogen on the uterus and breast tissue may exist when estrogen is administered systemically. Moreover, general administration may result in potential feminizing effects in males.

The local delivery of estrogen and the other compositions described above to atherosclerotic plaque is a promising alternative to the systemic use of this hormone. The basic anti-atherogenic properties of these compositions and their potential to inhibit neointimal proliferation while simultaneously attenuating endothelial repair make them ideal for local administration in the coronary artery to inhibit restenosis. Localized delivery of other compositions comprising sex hormones, anti-hormones, sex-hormone agonists, steroid-hormone inhibitors/antagonist (partial or full) or selective estrogen receptor modulators (SERMS), or combinations thereof, to the vasculature may prevent and treat vascular diseases such as stenosis, restenosis and atherosclerosis.

The local-delivery systems generally comprise a local-delivery device and at least one of the effective compositions described above. The compositions can be delivered locally to tissue, tubular organs, blood vessels, the coronary or peripheral of organs as well as to muscles (myocardium, skeletal or smooth muscles). The compositions can also be injected directly into the vessel, vessel wall or muscle.

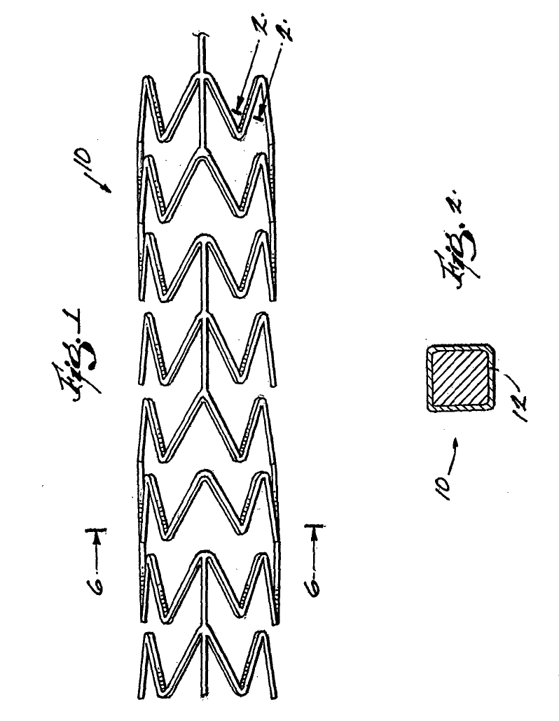

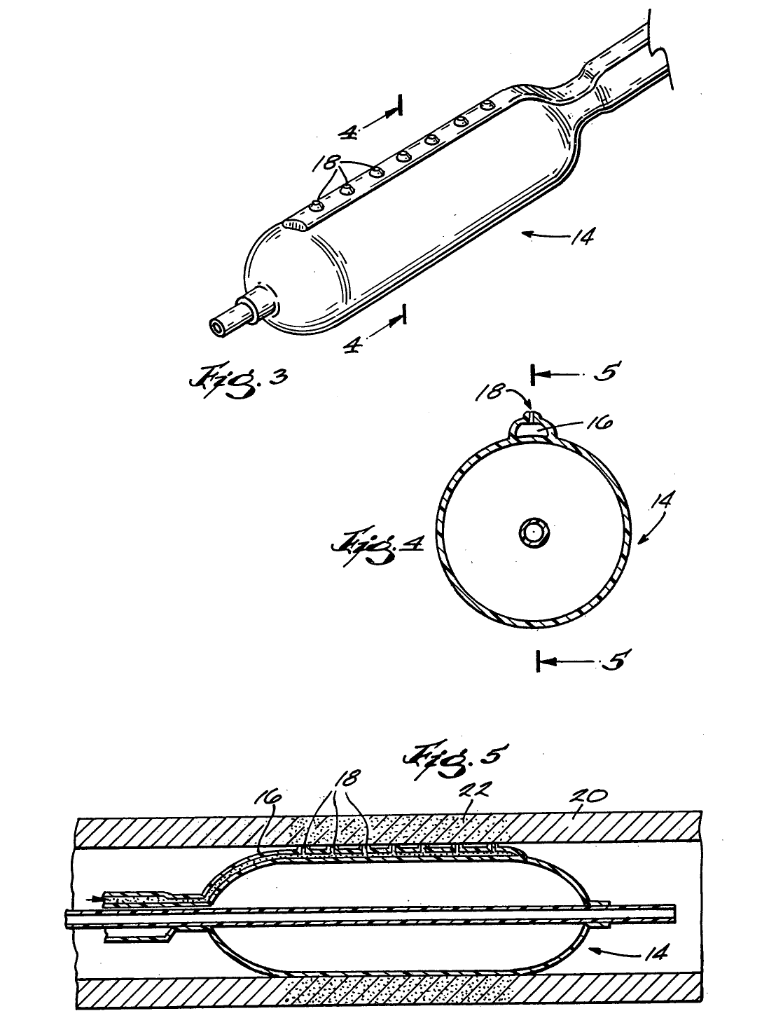

Examples of local-delivery devices include, but are not limited to, balloons, stents, catheters, wires and any other form of a local-delivery device. In one embodiment of the invention, the local-delivery system is a stent that delivers the above-described compositions to the localized portion of the body of a living organism. FIG. 1 illustrates a stent 10, which is a hollow member that lies within the lumen of a tubular structure and provides support and assures patency of an intact but contracted lumen. Stents may be made from stainless steel or any other suitable material such as biodegradable material (e.g. a Japanese stent). In other words, the stent itself may biodegrade. Effective compositions as described above coat or are applied to the stent. FIG. 2 shows a portion of the stent 10 coated with a composition 12 in cross-section. Because the stent remains in the artery after the angioplasty procedure is performed, it enables the composition 12 to slowly diffuse from the outside of its surface 10 into the adjacent atherosclerotic plaque to which it can affect. The rate of this diffusion varies according to the molecular weight of the compound being administered. Also, the structure of the stent and the type of coating applied thereto also affect the rate of diffusion.

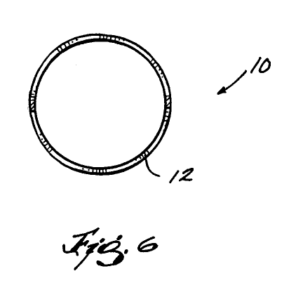

In another embodiment, an effective composition is applied to an injection catheter, and more particularly to a balloon-injection catheter 14. As shown in FIGS. 3 and 4, a balloon-injection catheter 14 is similar to a balloon angioplasty, except for the added feature of a chamber 16 including injection ports 18 for injecting the compositions described above. FIG. 5 illustrates a balloon-injection catheter 14 in cross-section after being injected into an affected area 20 of a living organism. The hormone can be injected directly into the plaque, vessel wall or tissue 22 via these injection ports 18. If an injection catheter injects the compound into the plaque 22, the composition releases immediately after injection. Accordingly, there is no residual release of the composition once the injection catheter is removed.

Angiographic, angioplasty, delivery and infusion catheters may also be used to deliver these compounds to affected areas. Using these devices, the above-described compositions can be locally delivered to a variety of body structures including grafts, saphenos vein grafts, arterial grafts, synthetic grafts, implants, prostheses or endoprostheses, homo or zeno grafts, cardiac muscle, skeletal or smooth muscle body structure.

Applying the Effective Compositions to the Local-Delivery Devices

Even a miniscule amount of composition may provide effective results. For example, at least about 1 μg, more particularly, greater than about 10 μg, even more particularly, greater than about 25 μg, and even more particularly, greater than about 50 μg may be used. There is no limit as to the maximum amount of composition that can be provided on the device, so long as the device is physically capable of holding the composition. In some examples, however, less than about 3000 μg of effective composition may be applied to each delivery device. More particularly, less than about 2000 μg, and even more particularly, less than about 1000 μg of effective composition may be applied to each delivery device. Effective dosages may widely vary; any dosage that restores circulation through a stenosed or restenosed blood vessel and/or alleviates the narrowing of the affected area is acceptable for use in the invention. As a result, dosages well in excess of the preferred ranges can be acceptable. The manner by which the effective compounds are bonded to the stent can also provide either slow or fast release of the effective compounds. Slow release of the effective compound can take up to ten years. Most preferably, release of the compound takes up to ten weeks, and more particularly, up to four weeks, although any period of time which allows for the effective compound to release from the stent or delivery device such that circulation is restored through the blood vessel and/or the narrowing of the affected area is alleviated is acceptable. Application of these effective compositions to a stent or other local-delivery device can be achieved in a number of different ways.

First, the compound can be mechanically, electromechanically, biologically, or chemically bonded to the delivery device, e.g. by a covalent bonding process. When using such a physical application the compounds are directly embedded into a metal or other suitable substance from which the local-delivery system is comprised.

Second, the effective composition can also be applied using a chemical coating/bonding process, whereby layers of a suitable pharmaceutical agent, vehicle, or carrier entrap the compound. In this manner, a biological or pharmacological coating already present on the local-delivery device acts as a platform for coating the compounds described above. Examples of platforms include, but are not limited to, silicon carbide, carbon, stainless steel, gold, nitinol, polymer absorbable platforms, diamond or diamond-like coating, e.g. polytetrafluoroethylene, hylauronic acid or polyactone. Other suitable synthetic pharmaceutical agents include, but are not limited to, phosphorylcholine, polyurethane, segmented polyurethane, poly-L-lactic acid, cellulose ester, polyethylene glycol as well as polyphosphate esters. Naturally occurring vehicles or carriers include collagens, laminens, heparins, fibrins, genes, DNA, proteins, vectors, viruses, and other naturally occurring substances that absorb to cellulose. Using a chemical coating of the stent or other device is particularly advantageous in that it allows the compound or sex hormone to slowly release from the carrier, vehicle, or agent. This extends the time that the affected portion of the body sustains the efficacious effects of the compounds. The manner in which these carriers or vehicles interact with the device material as well as the inherent structure of these carriers and vehicles provide a diffusion barrier, thereby controlling the release of the entrapped compounds or sex hormones. In other words, the manner by which the effective compounds are chemically bonded to the stent or delivery device can control slow or fast delivery of the compound.

Other suitable agents, vehicles, and carriers include polymers, elastomeric encapsulated non-erodable polymers (matrix release), elastomeric encapsulated erodable polymers (matrix release and conjugated release), nanoporous ceramic coatings, erodable polymer inlays, biopolymers, biologic graft materials, fibrin coatings and collagen coatings. The compositions may also be directly applied to the delivery devices.

Some examples of suitable agents, vehicles and carriers may be found in U.S. Pat. No. 6,344,035 issued to Chudzik on Feb. 5, 2002, U.S. Pat. No. 6,254,634 issued to Anderson et al. on Jul. 3, 2001, U.S. Pat. No. 6,214,901 issued to Chudzik et al. on Apr. 10, 2001, U.S. Pat. No. 6,121,027 issued to Clapper on Sep. 19, 2000, U.S. Pat. No. 5,464,650 issued to Berg on Nov. 7, 1995, and U.S. patent application Ser. No. 20020007215, Falotico, published on Jan. 17, 2002, and U.S. Pat. No. 6,113,613 issued to Spaulding on Sep. 5, 2000.

each of which is hereby fully incorporated by reference.

For example, a solvent, one or more complementary polymers dissolved in the solvent, and at least one of the above-identified effective compositions or agents dispersed in the polymer/solvent mixture may be prepared. The solvent may preferably be one in which the polymers form a true solution. The effective composition itself may either be soluble in the solvent or form a dispersion throughout the solvent.

The resultant composition can be applied to the device in any suitable fashion, e.g., it can be applied directly to the surface of the medical device, or alternatively, to the surface of a surface-modified medical device, by dipping, spraying, or any conventional technique. The method of applying the coating composition to the device is typically governed by the geometry of the device and other process considerations. The coating is subsequently cured by evaporation of the solvent. The curing process can be performed at room temperature, reduced or elevated temperature, or with the assistance of vacuum.

The polymer mixture may be biocompatible, e.g., such that it results in no induction of inflammation or irritation when implanted. In addition, the polymer combination must be useful under a broad spectrum of both absolute concentrations and relative concentrations of the polymers. This means that the physical characteristics of the coating, such as tenacity, durability, flexibility and expandability, will typically be adequate over a broad range of polymer concentrations. Furthermore, the ability of the coating to control the release rates of a variety of the above compositions can preferably be manipulated by varying the absolute and relative concentrations of the polymers.

A first polymer component may provide an optimal combination of various structural/finctional properties, including hydrophobicity, durability, bioactive agent release characteristics, biocompatability, molecular weight, and availability (and cost).

Examples of suitable first polymers include poly(alkyl)(meth)acrylates, and in particular, those with alkyl chain lengths from 2 to 8 carbons, and with molecular weights from 50 kilodaltons to 900 kilodaltons. A more specific example of a first polymer is poly n-butylmethacrylate. Such polymers are available commercially, e.g., from Aldrich, with molecular weights ranging from about 200,000 daltons to about 320,000 daltons, and with varying inherent viscosity, solubility, and form (e.g., as crystals or powder).

A second polymer component may provide an optimal combination of similar properties, and particularly when used in admixture with the first polymer component. Examples of suitable second polymers are available commercially and include poly(ethylene-co-vinyl acetate) having vinyl acetate concentrations of between about 10% and about 50%, in the form of beads, pellets, granules, etc. (commercially available are 12%, 14%, 18%, 25%, 33%). pEVA co-polymers with lower percent vinyl acetate become increasingly insoluble in typical solvents, whereas those with higher percent vinyl acetate become decreasingly durable.

A particularly preferred polymer mixture for use in this invention includes mixtures of poly(butylmethacrylate) (PBMA) and poly(ethylene-co-vinyl acetate) co-polymers (pEVA). This mixture of polymers has proven useful with absolute polymer concentrations (i.e., the total combined concentrations of both polymers in the coating composition), of between about 0.25 and about 70 percent (by weight). It has furthermore proven effective with individual polymer concentrations in the coating solution of between about 0.05 and about 70 weight percent. In one preferred embodiment the polymer mixture includes poly(n-butylmethacrylate) (PBMA) with a molecular weight of from 100 kilodaltons to 900 kilodaltons and a pEVA copolymer with a vinyl acetate content of from 24 to 36 weight percent. In a particularly preferred embodiment the a polymer mixture includes poly(n-butylmethacrylate) with a molecular weight of from 200 kilodaltons to 400 kilodaltons and a pEVA copolymer with a vinyl acetate content of from 30 to 34 weight percent. The concentration of the bioactive agent or agents dissolved or suspended in the coating mixture can range from 0.01 to 90 percent, by weight, based on the weight of the final coating composition.

Other suitable polymeric agents, vehicles and carriers may include, but are not limited to, at least one of polycarbonate, polyester, polyethylene, polyethylene terephthalate (PET), polyglycolic acid (PGA), polyolefin, poly-(p-phenyleneterephthalamide), polyphosphazene, polypropylene, polytetrafluoroethylene, polyurethane, polyvinyl chloride, polyacrylate (including polymethacrylate), and silicone elastomers, as well as copolymers and combinations thereof.

Similarly, other suitable polymeric agents, vehicles and carriers may include, but are not limited to, at least one of a synthetic polymer or copolymer selected from the group consisting of acrylics, vinyls, nylons, polyurethanes, polyethers, and biodegradable or bioerodable polymers selected from the group consisting of polylactic acid, polyglycolic acid, polydioxanones, polyanhydrides, and polyorthoesters.

Polymers may be synthetic or naturally occurring. Examples of synthetic polymers, include but are not limited to, oligomers, homopolymers, and copolymers resulting from addition or condensation polymerization. Naturally occurring polymers, such as polysaccharides and polypeptides, can be used as well.

Acrylic agents, vehicles and carriers may also be used. Such polymers may include hydroxyethyl acrylate, hydroxyethyl methacrylate, glyceryl acrylate, glyceryl methacrylate, acrylic acid, methacrylic acid, acrylamide and methacrylamide; vinyls such as polyvinyl pyrrolidone and polyvinyl alcohol; nylons such as polycaprolactam, polylauryl lactam, polyhexamethylene adipamide and polyhexamethylene dodecanediamide; polyurethanes; polyethers such as polyethylene oxide, polypropylene oxide, and polybutylene oxide; and biodegradable polymers such as polylactic acid, polyglycolic acid, polydioxanone, polyanhydrides, and polyorthoesters.

In addition, the device may be coated with a solution which includes a solvent, a polymer dissolved in the solvent and an effective amount of at least one of the compositions discussed above dispersed in the solvent. The solvent may be capable of placing the polymer into solution at the concentration desired in the solution. Examples of some additional suitable combinations of polymer, solvent and therapeutic substance are set forth below.

| POLYMER | SOLVENT |
|---|---|
| poly(L-lactic | chloroform |
| acid) | |
| poly(lactic | acetone |
| acid-co- | |
| glycolic acid) | |
| polyether | N-methyl |
| urethane | pyrrolidone |
| silicone | xylene |
| adhesive | |
| poly(hydroxy- | dichloro- |
| butyrate-co- | |
| methane | |
| hydroxyvalerate) | |
| fibrin | water |
| | buffered |
| | saline |

The solution is applied to the device and the solvent is allowed to evaporate, thereby leaving on the device surface a coating of the polymer and the effective composition. Typically, the solution can be applied to the device by either spraying the solution onto the device or immersing the device in the solution. Whether one chooses application by immersion or application by spraying depends principally on the viscosity and surface tension of the solution, however, it has been found that spraying in a fine spray such as that available from an airbrush will provide a coating with the greatest uniformity and will provide the greatest control over the amount of coating material to be applied to the device. In either a coating applied by spraying or by immersion, multiple application steps are generally desirable to provide improved coating uniformity and improved control over the amount of therapeutic substance to be applied to the device.

Preferably, the polymer is biocompatible and minimizes irritation to the vessel wall when the device is implanted. The polymer may be either a biostable or a bioabsorbable polymer depending on the desired rate of release or the desired degree of polymer stability, but a bioabsorbable polymer is probably more desirable since, unlike a biostable polymer, it will not be present long after implantation to cause any adverse, chronic local response. Bioabsorbable polymers that could be used include poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid. Also, biostable polymers with a relatively low chronic tissue response such as polyurethanes, silicones, and polyesters could be used and other polymers could also be used if they can be dissolved and cured or polymerized on the device such as polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The ratio of effective composition to polymer in the solution will depend on the efficacy of the polymer in securing the effective composition onto the device and the rate at which the coating is to release the effective composition to the tissue of the blood vessel. More polymer may be needed if it has relatively poor efficacy in retaining the therapeutic substance on the device and more polymer may be needed in order to provide an elution matrix that limits the elution of a very soluble therapeutic substance. A wide ratio of therapeutic substance to polymer could therefore be appropriate and could range from about 10:1 to about 1:100.

Estrogen and the other effective compositions described above can also be coated onto or delivered with other drugs or compounds in order to administer synergistic treatment. Examples of other suitable drugs and compounds include antibodies, oligonucleotides (e.g. antisense oligonucleotides), antiproliferatives, anticancer or antimicrotubular agents (e.g. rapamycin, paclitaxel), antiproliferative agents, growth factors, genes, antisense or antithrombotic agents or any other chemical or biological compound that will act synergistically to increase the effectiveness of the primary hormone or compound. Additional agents include the following: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, anti-lipid agents, inhibitors of surface glycoprotein receptors, antiplatelet agents, antimitotics, microtubule inhibitors, anti secretory agents, actin inhibitors, remodeling inhibitors, antisense nucleotides, anti metabolites, anticancer chemotherapeutic agents, anti-inflammatory steroid or non-steroidal anti-inflammatory agents, immunosuppressive agents, growth hormone antagonists, dopamine agonists, radiotherapeutic agents, peptides, proteins, enzymes, extracellular matrix components, angiotensin-converting enzyme (ACE) inhibitors, free radical scavengers, chelators, antioxidants, anti polymerases, antiviral agents, photodynamic therapy agents, and gene therapy agents.

For example, stent coatings can absorb and release these materials, thus providing an inert depot for controlled drug administration. Loading of the drug can occur for example via diffusion of the drug solution into the coating by hydration/swelling of the polymer matrix. The bioactive (e.g., pharmaceutical) agents useful in the present invention include virtually any therapeutic substance which possesses desirable therapeutic characteristics for application to the implant site.

In addition to providing methods for treating and preventing, among other things, stenosis and restenosis, the invention also provides methods of treating or preventing methods of treating or preventing high-risk plaque. “High-risk plaque” includes, but is not limited to, vulnerable plaque, atherosclerotic plaque, ruptured plaque, activated plaque, non-critical lesions, as well as plaque that could possibly rupture or become vulnerable or activated (regardless of how small the possibility). The invention provides methods of treating a plaque that has been determined to be susceptible to subsequent rupture and/or sudden progression. As used herein, the term “vulnerable plaque” is meant to refer to plaque that has the propensity or is prone to rupture or become active and attract platelets, fibrin, thrombin and other coagulation factors to cause thrombosis. Plaque erosions or ruptures can cause acute coronary syndromes.

Plaques prone to rupture are characterized by a large lipid core and a thin fibrous cap, but plaques with erosion vary in size and composition. Inflammatory activity has been associated with plaque erosion and may have a role in the pathogenesis of endothelial damage. Erosions and subsequent thrombosis can develop in plaques that are relatively rich in proteoglycan matrix and smooth-muscle cells and that lack a superficial lipid core. Plaque rupture may result from intrinsic plaque vulnerability, mechanical stresses, and extrinsic triggers. A plaque with a thin fibrous cap overlaying a large lipid core is a high risk for rupture.

Vulnerable plaques may have a well-preserved lumen, because plaques grow inwardly initially, as well as the substantial lipid core, and the thin fibrous cap separating the tissue factor. The lipid-rich core may be in the central portion of the eccentrically thickened lumina. The fibrous cap, composed mainly of connective tissue, may be on the luminal side of the lipid core. This fibrous cap may be the only barrier separating the circulation, and its powerful coagulation system designed to generate thrombus, from the lipid core, a highly thrombogenic material rich in tissue factor, one of the most potent procoagulants known. At the edges of the fibrous cap overlying this lipid core is the shoulder region, enriched with macrophages and lipid-laden macrophage-derived foam cells. These lesional macrophages and foam cells produce a variety of substances, including tissue factor bearing thrombogenic macrophages from the blood. Smooth muscle cells (SMCs) are often activated at sites of lesion disruption. The following indicates some additional characteristics of vulnerable plaque: increased numerous cells of inflammatory cells (e.g., macrophages and T cells); the thin fibrous cap separating the circulation from procoagulants in the plaque lipid core; and a relative paucity of vascular smooth muscle cells (VSMC).

In contrast, stable plaques have relatively thick fibrous caps protecting the lipid core from contact with the blood. Stable plaques are often more detectable but may also be indistinguishable at angiography compared to vulnerable plaques. With stable plaque, the thickness and integrity of the fibrous cap overlying the lipid-rich core is a principal factor in the stability of the plaque. Plaque stability may be a function of some of the following dynamic factors: VSMC production of the extracellular matrix that is the bulwark of the fibrous cap, interaction of inflammatory cells, inhibition of this process by certain cytokines, and increased degradation of the matrix by matrix metalloproteinases.

The composition and vulnerability of plaque may play one of the primary roles in determining the development of thrombus mediated acute coronary events. Rupture at the site of a vulnerable atherosclerotic plaque may be one of the most frequent causes of acute coronary syndromes. Typically, such plaque does not cause high-grade stenosis and has a large lipid core and a thin fibrous cap that is often infiltrated by inflammatory cells. Plaque rupture usually leads to various degrees of thrombus formation. Vulnerable atherosclerotic plaque may not always cause high-grade stenosis, however, it may result in an acute coronary syndrome, such as unstable angina, myocardial infarction, or in worse cases, sudden death.

Vulnerable plaque may be identified using a variety of techniques that are well-known in the art. Well-known techniques such as thermagraphy, spectroscopy, radioisotope scinography, use of inflammatory serum markers, intravascular ultrasonography, electron-beam computed tomography, angioscopy, instravascular ultrasound, and magnetic resonance imaging may be used.

The following articles provide more background regarding vulnerable plaque, and are both hereby fully incorporated by reference: Plutzky, Jorge MD, Atherosclerotic Plaque Rupture: Emerging Insights and Opportunities, The American Journal of Cardiology, Vol. 84 (1A), (July, 1999), as well as Kullo et al., Vulnerable Plaque: Pathobiology and Clinical Implications, Annals of Internal Medicine, Vol. 129, No. 12 (1998).

The devices discussed above can be used to treat the vulnerable plaque. More particularly, an effective dose of one of the compositions set forth above may be applied to one of the devices discussed above (e.g., a self-expanding or balloon-expandable stent). The device is inserted into an area of a living organism affected by the vulnerable plaque in order to treat or prevent the same. The device may or may not directly contact the affected area, however, the device allows for the gradual release of the composition therefrom in order to treat or prevent the plaque. In one embodiment, a stent or other device is at least partially coated with a platform, carrier or agent, which at least partially encompasses an effective dose of a composition comprising estrogen, estradiol, or a derivative thereof. The stent is inserted into an area of the body affected by the vulnerable plaque, and the effective dose is allowed to gradually release, thereby treating or preventing the vulnerable plaque. Similarly, the invention may be used to prevent the progression of atherosclerosis.

### EXAMPLE 1

In one preferred example, powdered or liquid estrogen is mixed with a carrier such as ethanol to form a solution or gel. The estrogen gel is then applied to a stainless steel stent using chemical coating methods that are well-known in the art. Subsequently, the coated stent is inserted into an arterial lumen of a human being suffering from atherosclerosis. In other words, the coated stent is inserted into an artery plagued by patchy, intramural plaque. The estrogen in the coating slowly diffuses into and penetrates the plaque, thereby providing treatment for this vascular disease.

### EXAMPLE 2

In another example, low and high dose 17B-estradiol eluting stents were compared with control stents in a randomized fashion in 18 porcine coronary arteries. Each artery of six pigs were randomly stented with either a control, low-dose or high-dose 17-estradiol eluting stent. All animals were sacrificed at 30 days for histomorphometric analysis.

### Animal Preparation

The experiment and animal care conformed to National Institutes of Health and American Heart Association guidelines for the care and use of animals and were approved by the Institutional Animal Care and Use Committee at the Washington Hospital Center. Six domestic juvenile swine weighing 35-45 kg were used. They were premedicated with acetylsalicyclic acid 350 mg for a day prior and 75 mg of clopidogrel for 3 days prior to the procedure and until sacrifice. The swines were sedated with a combination of ketamine (20 mg/kg) and xylazinc (2 mg/kg), by intramuscular injection. They were given pentobarbital (10-30 mg/kg IV), and were subsequently intubated and ventilated with oxygen (2 L/min) and isoflurane 1% (1.5 L/min). An 8F-introducer sheath was inserted into the right carotid artery by surgical cut down. Heparin (150 units/kg) was administered intra-arterially. Heart rate, blood pressure and electrocardiography were monitored throughout the procedure.

### Protocol for Loading of 17B-Estradiol onto Stents

Two-doses of 17B-estradiol powder (100 mg, dissolved in ethanol {5.0 ml}, Sigma, St. Louis, Mo.) were impregnated onto phosphorylcholine (PC) coated stainless steel stents (BiodivYsio™DD Stent {3.0 mm×18 mm}, Biocompatibles Ltd., Surrey, United Kingdom). The stents were immersed into the estradiol solution for 5 minutes and then allowed to dry at room temperature for another 5 minutes. For the high dose stent, a 10 μl aliquot of solution was pipette onto the stent and spread instantly and diffused into the stent. After being allowed to dry for 1 minute, this step was repeated and the stent was allowed to dry for 10 minutes prior to implantation. In vitro studies indicate that an estradiol dose of 67 μg (range: 51-88 μg) for the low dose stent and 240 μg (range: 229-254 μg) for the high dose can be loaded onto a 3.0×18 mm stent.

### Stent Deployment

Coronary angiography was performed after intracoronary nitroglycerin (200 μg) administration and recorded on cine film (Phillips Cardiodiagnost; Shelton, Conn.). Using high-pressure dilatation (12-14 atm×30 sec), a single stent of each type was deployed in all 3 coronaries of each animal in a randomized fashion so that the 3 different types of stents were deployed in a different artery for each pig. The operator was blinded to the stent type being deployed. The stent artery ratio was kept between 1:1.3 and 1:1.2. All animals tolerated the stenting procedure and survived until 30 days after which they were sacrificed and the hearts were perfusion-fixed.

### Quantitative Histomorphometric Analysts

The histopathologist was blinded to the stent types in each artery. Cross sections of the stented coronary arteries were stained with metachromatic stain (Stat Stain for Frozen Sections, Eng. Scientific, Inc., 82 Industrial Fast, Clifton, N.J., 07012), Area measurements were obtained by tracing the external elastic lamina (vessel area, VA, mm²) stent line (stent strut area, mm²) lumen perimeter (luminal area, LA, mm²) and neointimal perimeter (intimal area, IA, mm²). The vessel injury score was determined by the method described by Kornowski et al. The scoring of endothelialization is based on percent of intimal surface covered by endothelial cells. 1+ equals less than ¼ of the intimal surface is covered by endothelial cells, 2+ equals over ¼ and less than ¾ covered and 3+ equals greater than ¾ to complete coverage of the intimal surface.

### Statistical Analysis

Data (mean±standard deviation) were analyzed to determine differences between treatment groups using an ANOVA with a post-hoc Bonferroni analysis. Comparison of the mean values with a p-value of less than 0.05 was considered statistically significant.

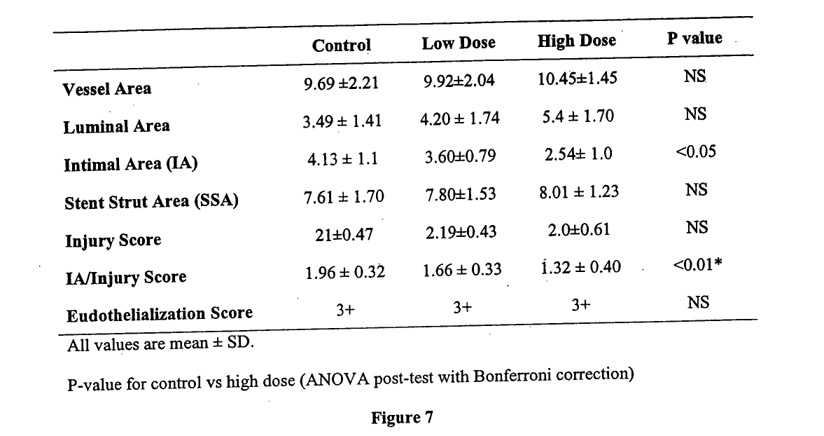

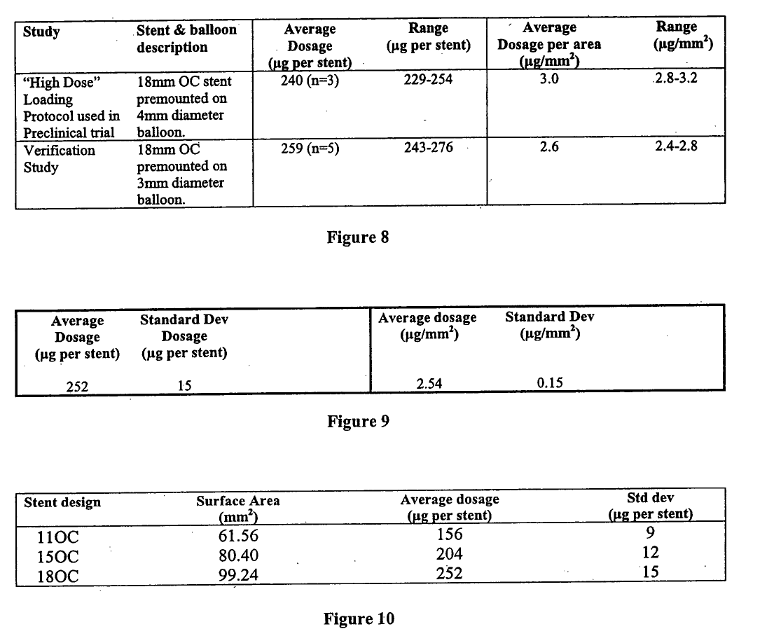

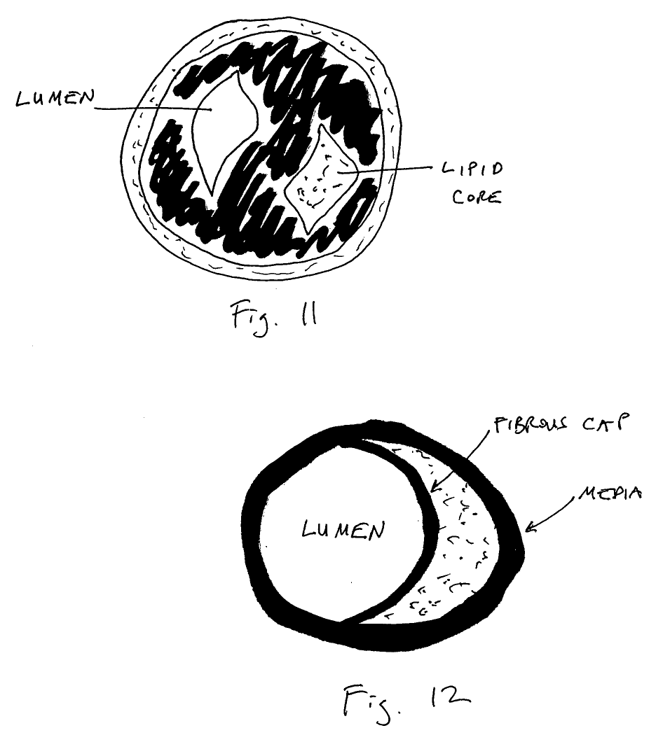

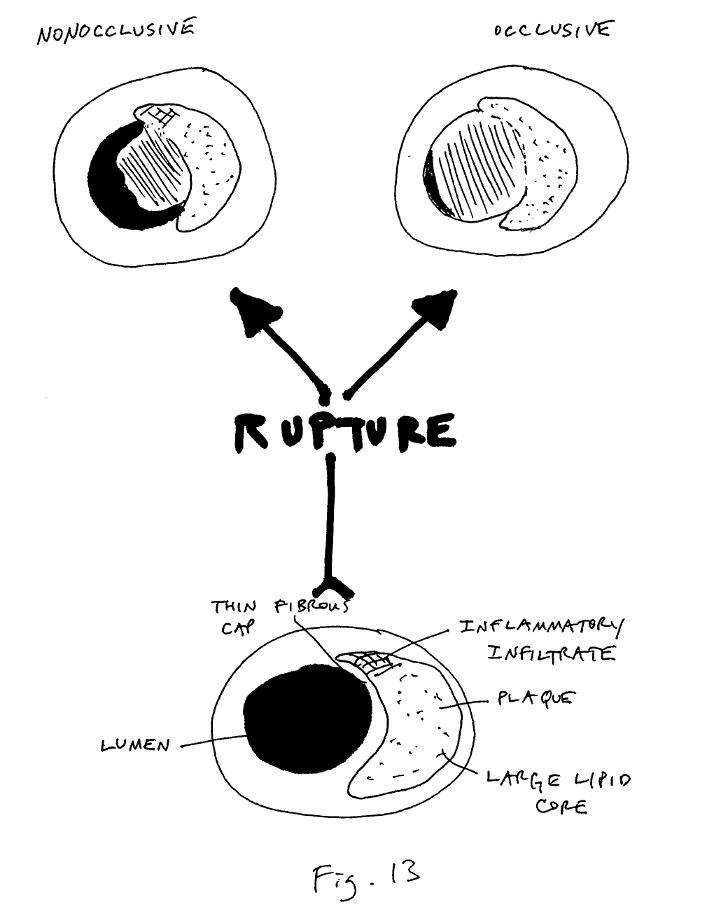

There was a 40% reduction in intimal area in the high dose stents compared with control stents (2.54±1.0 mm² vs 4.13±1.1 mm², for high dose vs control respectively, P<0.05. see Table 1.). There was also a reduction in the IA/Injury score ratio in the high dose group compared with the control stents (1.32±0.40 mm² vs 1.96±0.32 mm², for high dose vs control respectively, P<0.01, see Table 1.). FIG. 7a) illustrates the histological appearance of the control stented segments at 30 days. FIG. 7b) illustrates the histological appearance of the low-dose stented segments at 30 days and FIG. 7c) illustrates the histological appearance of the high dose stented segments at 30 days. More than ¾ to complete coverage with endothelium was observed in all 3 groups (endothelialization score=3+). There was 3+ endothelialization score observed in all the stent groups.

This is the first study to show that 17B-estradiol eluting stents reduce intimal proliferation without effecting endothelial regeneration in the pig model of instent restenosis. Estrogen coated stents prevent and treat instent restenosis.

The basic anti-atherogenic properties of estrogen with the potential to inhibit neointimal proliferation whilst not effecting endothelial repair appears to make estrogen an ideal compound to be delivered on a stent. Previous research has shown that a single intracoronary infusion of estrogen can inhibit smooth muscle cell proliferation in the pig after angioplasty.

The pathophysiology of restenosis involves neointimal hyperplasia and negative vessel remodeling. Although the low dose 17B-estradiol stents only demonstrated a trend towards a reduction in intimal area, the high dose 17B-estradiol stents significantly inhibited the neo-intimal proliferative response by about 40% compared with control stents.

One of the major limitations of current therapies for restenosis such as brachytherapy is that of late stent thrombosis. A delay in re-endothelialization causing a persistent thrombogenic coronary surface is the most plausible explanation for this side effect. There was no evidence of inhibition of endothelial cell regeneration in the low or high dose stented arteries compared with control.

These 2 findings were observed with the use of a relatively low systemic dose of estrogen. Systemic doses usually range between 25-30 μg/kg, which is more than 2-3 the total dose of estrogen loaded onto the high dose stent. In fact, many clinical studies have acutely administered higher doses (systemically or intracoronary) in both male and females with no untoward effects. If hypothetically the entire, high dose (264 μg), were eluted from the stent into the systemic circulation as a single bolus, no side effects would be expected. The delivery of a relatively low dose of estrogen directly on a stent to inhibit restenosis without impeding endothelial regeneration represents a major theoretical advantage over radiation therapy and perhaps other locally delivered anti-proliferative drugs.

Consequently, this demonstrate that estrogen impregnated stents reduce the intimal proliferative response to stent implantation without impeding re-endothelialization. Since 17B-estradiol is an endogenous circulating hormone in both males and females, in this relatively low systemic dose, it may provide a simple, non-toxic therapy for treating de novo coronary lesions, small vessels and diffise disease.

### EXAMPLE 3

Coronary stent implantation has been proven superior to conventional balloon angioplasty for the treatment of coronary de-novo lesions. However, coronary stenting procedures are still burdened with an unacceptable high restenosis rate. The utilization of antiproliferative agents delivered locally via drug-eluting stents has dramatically reduced these rates. However, as in the case of brachytherapy, concern remains regarding delayed healing of the arterial wall and the long-term effects of cell-cycle inhibitors. An alternative approach for the prevention of in-stent restenosis involves the use of a naturally occurring vasculoprotective hormone such as 17β-Estradiol. 17β-Estradiol has a low molecular weight, is hydrophobic and lipophilic making it pharmacokinetically suitable for loading on a stent delivery system. Example 2 suggests that the local delivery of 17β-Estradiol either via an infusion catheter or impregnated on a stent inhibits neointimal proliferation without affecting endothelial repair and function.

### Methods

This was a single-center prospective trial of 30 patients who were scheduled to undergo elective percutaneous intervention for single, short (<18mm in length), de novo lesions in native coronary arteries with 2.5-3.5 mm in diameter. All patients received aspirin (325 mg/d, indefinitely) at least 12 hours before the procedure, and clopidogrel (300 mg at least 6 hours prior to stent implantation and 75 mg daily continued for 60 days). All patients underwent angiographic and IVUS follow-up at 6 months. The patients returned for clinical visits at 30 days, 6 and 12 months in which physicians were blinded to the angiographic and ultrasonographic data. The protocol was approved by the Medical Ethics Committee of the Institute Dante Pazzanese of Cardiology, and informed consent was obtained from every patient.

### Loading 17β-Estradiol Stents

The BiodivYsio stent delivery system (Biocompatibles Ltd, United Kingdom) is a laser cut, 316L stainless steel balloon-expandable stent coated with phosphorylcholine (PC), a naturally occurring biological substance. The biocompatible PC coating constitutes a 50-100 nm thick double layer of synthetic PC coating that is able to adsorb a drug via a “sponge-like” mechanism. The method of impregnating the PC coating involves 3 steps: First, immersing the stent into a solution of 17β-Estradiol (in ethanol) for 5 minutes. After removal of the stent from the solution and allowing it to dry for 1 minute, a second step whereby 10 μl of the same solution is pipetted onto the stent. The PC polymer absorbs the solution like a sponge. The stent is again allowed to air dry for 1 minute. This process is repeated, but with 5 minutes of air-drying (total preparation time=12 minutes). The stent is then immediately deployed. Laboratory testing has demonstrated a consistent amount of drug (2.52 μg/mm²) can be impregnated using this method.

### Procedure

Each patient received one 18 mm stent (3.0 to 3.5 mm in diameter). All lesions were pre-dilated. Stents were deployed at high-pressure (>14 atm) and the need for post-dilatation was guided by intravascular ultrasound (IVUS).

### Ouantitative Measurements

Baseline, post-procedure and 6-month follow-up quantitative coronary angiography (QCA) analysis were performed in all patients, by an independent core-laboratory (Cardiovascular Imaging Core Laboratories, University of Florida, Jacksonville, USA). Quantitative measurements of the in-stent and in-lesion (in-stent segment plus 5 mm edge proximally and distally) segments were performed in 2 orthogonal projections. Intravascular ultrasound (IVUS) imaging was performed in all patients post-procedure and at follow-up. IVUS images were acquired using motorized pullback at a constant speed of 0.5 mm/s (Galaxy, Boston Scientific, Natick, Mass.). Three-dimensional IVUS volumetric analysis was performed by an independent core laboratory (Cardiovascular Imaging Core Laboratories, University of Florida, Jacksonville, USA). Percent volume obstruction was defined as the ratio of the volume of neointimal hyperplasia to the volume of the stent multiplied by 100.

### Statistical Analysis

Statistical analysis was performed with the aid of the commercially available software (SPSS version 11). Quantitative data are presented as rates or mean value±SD. Probability values are 2-sided from Student's t test for continuous variables and Fisher's exact test for categorical variables. A value of P<0.05 was considered significant.

### Results

The mean age of the patients was 61±12 years. A total of 21 patients (70%) were males. Systemic hypertension was the most frequent coronary risk factor, involving 15 patients (49%), followed by smoking in 10 patients (33%) and dislipedemia in 8 (27%) whereas only 3 patients (10%) were diabetics. Eleven patients (37%) had a prior history of myocardial infarction (MI). The procedure was successful in all patients. There were no in-hospital events including no elevation of cardiac enzymes post-procedure. One patient underwent target lesion revascularization at 6-month follow-up due to symptomatic angiographic restenosis. All other patients were asymptomatic at 6-month angiographic follow-up. There was no stent thrombosis or other MACE (major cardiovascular events including death, MI, stroke or target vessel revascularization) up to 12-month clinical follow-up.

### Aniographic Follow-Up

Mean lesion length was 9.1±2.4mm. Two patients developed in-stent restenosis (>50% diameter stenosis). One patient with a 60% lesion was asymptomatic with negative non-invasive stress test and did not undergo repeat revascularization. There was no restenosis at the stent edge segments, and in-segment late loss was only 0.34 mm.

### Six-month IVUS Analysis

The neointimal hyperplasia volume amounted to 32.3±16.4 mm³ with the stent volume of 143.7±43.7 mm³, resulting in a mean neointimal volume obstruction of 23.5±12.5%. No patient had ≧50% volume obstruction by IVUS. There was no evidence of stent malapposition or echolucent images (“black-hole”).

### Discussion

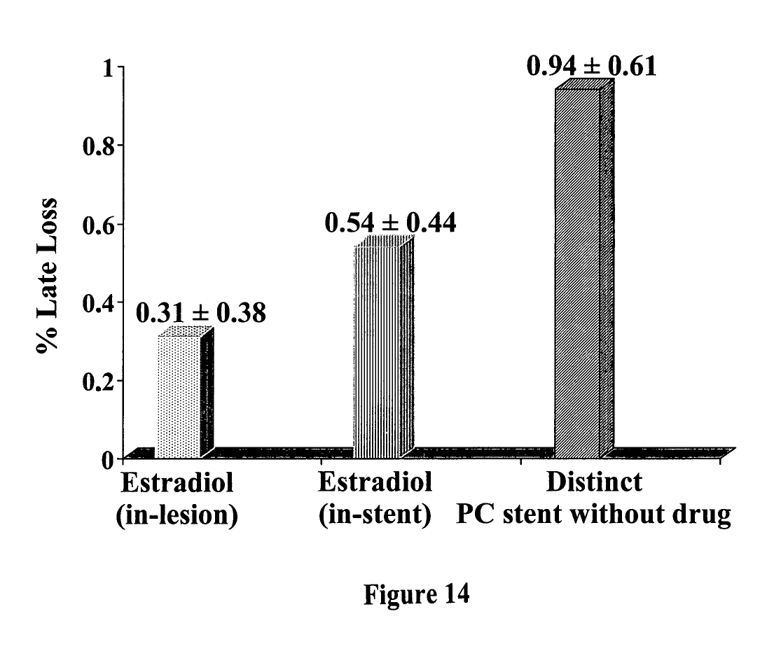

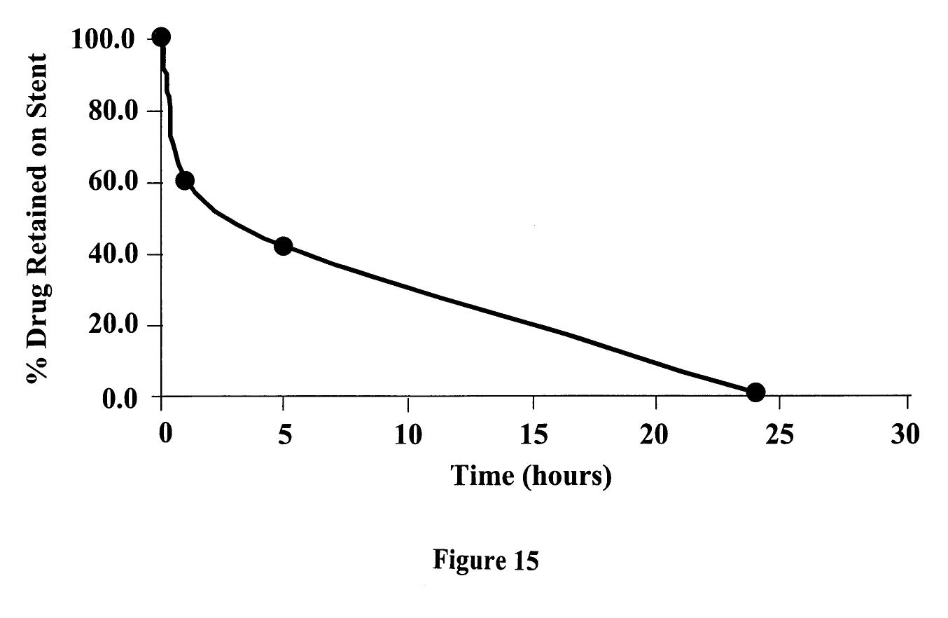

This study is the first human experience with 17β-Estradiol eluting stents for the prevention of restenosis. Clinical outcomes up to 1-year follow-up suggest that the use of 17β-Estradiol PC-coated eluting stents is safe and feasible, with a low incidence of restenosis and without associated local or systemic toxicity. Only 1 (out of 30) patients required target vessel revascularization. The angiographic and IVUS follow-up results at 6 months demonstrated a low amount of intimal hyperplasia and late-loss, which compares favorably with previous studies testing the same PC coated BiodivYsio stents without estradiol (FIG. 14). In addition, there was minimal in-segment late-loss and no edge restenosis. Nevertheless, neointimal proliferation was not completely abolished by estradiol-eluting stents. Estradiol eluting from “hand” loaded PC coated stents is only carried-out within the first 24 hours interval (FIG. 15). Nonetheless, the amount of intimal hyperplasia detected by IVUS compares favorably with bare metal stents suggesting an anti-restenotic effect of estradiol in spite of the suboptimal stent elution.

Estradiol can inhibit smooth muscle cell proliferation and migration, accelerate re-endothelialization and restore normal endothelial function following balloon artery injury. Inhibition of neointimal proliferation and accelerated re-endothelialization and function with the injection of 17β-Estradiol following balloon angioplasty in a pig model is shown in Example 2. In addition, pre-clinical work with the same stent, dose and loading process of 17β-estradiol as in this Example, suggests a 40% reduction in in-stent neo-intimal formation. Estradiol is known to have pleomorphic properties. It has anti-atherogenic, anti-inflammatory and anti-oxidant properties as well as a wide therapeutic window. These features may contribute to its vasculoprotective effect and may also make it a potential agent in the treatment of the vulnerable plaque.

There was no stent thrombosis despite short duration of antiplatelet therapy in the present study. In addition, late stent malapposition was not detected by IVUS and late loss at the stent margins (in-segment analysis) was minimal while no edge restenosis was found at 6-month follow-up. Hence, an adequate safety profile of 17-estradiol eluted stent was demonstrated in this Example.

This is the first study in humans to demonstrate that 17β-Estradiol eluting stents are feasible and safe. Low rates of restenosis and revascularization were observed. At 1-year follow-up, these results appear to be sustained. These seminal observations suggest that vasculoprotective agents such as estradiol may provide an alternative approach to anti-proliferative agents in the prevention of restenosis and warrant further investigation with a large, randomized multicenter trial.

1. A method of treating or preventing high-risk plaque, the method comprising:
applying to a medical device an effective amount of a composition comprising a sex hormone, anti-hormone, sex-hormone agonist, steroid-hormone inhibitor/antagonist (partial or full), selective estrogen receptor modulator (SERM), or a combination thereof; and
inserting the medical device into an area of a living organism that is or has a propensity to be affected by high-risk plaque.

2. The method of claim 1, further comprising allowing at least a portion of the sex hormone, anti-hormone, sex-hormone agonist, steroid-hormone inhibitor/antagonist (partial or full), selective estrogen receptor modulator (SERM), or combination thereof, to gradually release from the medical device into the area of the living organism that is or has a propersity to be affected by the high-risk plaque, thereby treating or preventing the high-risk plaque.

3. The method of claim 1, wherein the composition comprises a sex hormone and the sex hormone comprises estrogen, progesterone, testosterone, dehydroepiandrostrone (DHEA), dehydroepiandrosteronesulfate (DHEA) or a combination thereof.

4. The method of claim 1, wherein the medical device comprises a stent.

5. The method of claim 1, wherein the medical device comprises a catheter, a balloon catheter or a balloon.

6. The method of claim 1, wherein the composition comprises a sex hormone and the sex hormone comprises estrogen.

7. The method of claim 6, wherein the medical device comprises a stent.

8. The method of claim 6, wherein the medical device comprises a catheter.

9. The method of claim 1, wherein the composition comprises a sex-hormone agonist and the sex-hormone agonist comprises estradiol, estrone, ethinyl estradiol, conjugated equine estrogen, or a combination thereof.

10. The method of claim 1, wherein the composition comprises an anti-hormone and the anti-hormone comprises an anti-estrogen, Faslodex, an anti-androgen, cyproterone acetate, an anti-testosterone, or a combination thereof.

11. The method of claim 1, wherein the composition comprises a steroid-hormone inhibitor/antagonist (partial or full) and the steroid-hormone inhibitor/antagonist (partial or full) comprises aminogluthemide, anastrazole, letrozole, or a combination thereof.

12. The method of claim 1, wherein the composition comprises a SERM and the SERM comprises a raloxifene, tamoxifen, tibolone, idoxifene, or a combination thereof.

13. The method of claim 1, wherein the affected area of the living organism comprises tissue, tubular organs, blood vessels, coronary or peripheral of organs, myocardium, skeletal, smooth muscles or a combination thereof.

14. The method of claim 1, wherein the effective dose of the composition comprises about 50 μg to about 1000 μg.

15. The method of claim 1, wherein the effective dose of the composition comprises about 50 μg to about 276 μg.

16. The method of claim 1, wherein the composition further comprises an antibody, oligonucleotide, antiproliferative, anticancer agent, growth factor, gene, antithrombotic agent thrombin inhibitor, antithrombogenic agent, thrombolytic agent, fibrinolytic agent, vasospasm inhibitor, calcium channel blocker, vasodilator, antihypertensive agent, antimicrobial agent, antibiotic, anti-lipid agent, inhibitor of surface glycoprotein receptors, antiplatelet agent, antimitotic, microtubule inhibitor, anti-secretory agent, actin inhibitor, remodeling inhibitor, antisense nucleotide, anti-metabolite, anticancer chemotherapeutic agent, anti-inflammatory steroid or non-steroidal anti-inflammatory agent, immunosuppressive agent, growth hormone antagonist, dopamine agonist, radiotherapeutic agent, peptide, protein, enzyme, extracellular matrix component, angiotensin-converting enzyme (ACE) inhibitor, free radical scavenger, chelator, antioxidant, anti-polymerase, antiviral agent, photodynamic therapy agent, gene therapy agent or combination thereof.

17. The method of claim 1, wherein the high-risk plaque comprises vulnerable plaque.

18. A local-delivery device for treating or preventing high-risk plaque in a living organism, the device comprising: a medical device at least partially coated with an effective dose of a composition comprising a sex hormone, anti-hormone, sex-hormone agonist, steroid-hormone inhibitor/antagonist (partial or full), selective estrogen receptor modulator (SERM), or a combination thereof, the local-delivery device being suitable for treating or preventing high-risk plaque.

19. The device of claim 18, wherein the medical device comprises a stent.

20. The device of claim 19, wherein the composition comprises a sex hormone, and the sex hormone comprises estrogen.

21. The device of claim 18, wherein the medical device comprises a catheter, a balloon catheter or a balloon.

22. The device of claim 18, further comprising a platform, natural carrier, pharmaceutical agent, polymer or combination thereof at least partially encompassing the composition, thereby allowing for gradual release of the composition therefrom when the medical device is inserted into a living organism.

23. The device of claim 18, wherein the medical device is at least partially coated with a polymer and the polymer comprises a biostable polymer, a bioabsorbable polymer, biodegradable, bioerodable or a combination thereof.

24. The device of claim 18, wherein the effective dose of the composition comprises about 50 μg to about 1000 μg.

25. The device of claim 18, wherein the effective dose of the composition comprises about 50 μg to about 276 μg.

26. The device of claim 18, wherein the effective dose of the composition comprises about 2.4 μg to about 3.2 μg per 1 mm² of medical device.

27. The device of claim 18, wherein the composition further comprises a sex hormone, anti-hormone, sex-hormone agonist, steroid-hormone inhibitor/antagonist (partial or full), selective estrogen receptor modulator (SERM) or a combination thereof.

28. The device of claim 18, wherein the composition further comprises an antibody, oligonucleotide, antiproliferative, anticancer agent, growth factor, gene, antithrombotic agent thrombin inhibitor, antithrombogenic agent, thrombolytic agent, fibrinolytic agent, vasospasm inhibitor, calcium channel blocker, vasodilator, antihypertensive agent, antimicrobial agent, antibiotic, anti-lipid agent, inhibitor of surface glycoprotein receptors, antiplatelet agent, antimitotic, microtubule inhibitor, anti-secretory agent, actin inhibitor, remodeling inhibitor, antisense nucleotide, anti metabolite, anticancer chemotherapeutic agent, anti-inflammatory steroid or non-steroidal anti-inflammatory agent, immunosuppressive agent, growth hormone antagonist, dopamine agonist, radiotherapeutic agent, peptide, protein, enzyme, extracellular matrix component, angiotensin-converting enzyme (ACE) inhibitor, free radical scavenger, chelator, antioxidant, anti polymerase, antiviral agent, photodynamic therapy agent, gene therapy agent or combination thereof.

29. A method of treating high-risk plaque in a living organism, the method comprising:
applying an effective dose of a composition comprising estrogen, estradiol or a derivative thereof to a stent by chemical or physical bonding;
placing the stent at or near high-risk plaque; and
releasing the estrogen, estradiol or derivative thereof.

30. The method of claim 29, wherein applying an effective dose of the composition to the stent comprises immersing the stent in a solution comprising estrogen, estradiol or a combination thereof and allowing the stent to dry.

31. The method of claim 29, wherein the effective dose of the composition comprises about 50 μg to about 1000 μg.

32. The method of claim 29, wherein the effective dose of the composition comprises about 50 μg to about 276 μg.

33. The method of claim 29, wherein the effective dose comprises about 2.4 μg to about 3.2 μg of the composition per 1 mM² of stent.

34. The method of claim 29, wherein the chemical bonding comprises at least partially encompassing the composition with a platform, natural carrier, pharmaceutical agent, polymer or combination to allow for gradual elution of the composition therefrom.

35. The method of claim 34, wherein the platform at least partially encompasses the composition and the platform comprises silicon carbide, carbon, diamond, diamond-like coating, polytetrafluoroethylene, hylauronic acid, polyactone or a combination thereof.

36. The method of claim 34, wherein the natural carrier at least partially encompasses the composition and the natural carrier comprises collagen, laminen, heparin, fibrin, a naturally occurring substance that absorbs to cellulose or a combination thereof.

37. The method of claim 34, wherein the pharmaceutical agent at least partially encompasses the composition and the pharmaceutical agent comprises polyurethane, segmented polyurethane, poly-L-lactic acid, cellulose ester, polyethylene glycol, polyphosphate esters or a combination thereof.

38. The method of claim 34, wherein the polymer at least partially encompasses the composition and the polymer comprises a biostable polymer, a bioabsorbable polymer or a combination thereof.

39. The method of claim 29, wherein the high-risk plaque comprises vulnerable plaque.
